(19) United States
(12) Patent Application Publication
(10) Pub. No.: US 2006/0203196 A1
(43) Pub. Date: Sep. 14, 2006

(54) INTERACTIVE REFRACTOR INCORPORATING WAVEFRONT SENSING AND ADAPTIVE OPTICS

(76) Inventor: Anthony Y. Van Heugten, Sarasota, FL (US)

Correspondence Address: PERKINS COIE LLP, POST OFFICE BOX 1208, SEATTLE, WA, 98111-1208, US

(21) Appl. No.: 11/421,525

(22) Filed: Jun. 1, 2006

Related U.S. Application Data
(63) Continuation of application No. PCT/US04/40425, filed on Dec. 2, 2004.
(60) Provisional application No. 60/526,176, filed on Dec. 2, 2003.

(51) Int. Cl. A61B 3/10

(52) U.S. Cl. 351/221

(57) ABSTRACT

An integrated wavefront sensor and adaptive optic system includes an electroactive lens (40) positioned such that light from real world objects (42) being observed by a patient is combined, by a combiner optic (44), with a wavefront sensor illumination beam (12) into a single beam of light (46) that passes through or onto the electroactive lens (40). With this integrated system, the patient's vision may be measured and corrected, taking into account changes in the higher order aberrations that are influenced by the patient's accommodation, simultaneous to the patient observing, in real time, the refractive correction being proposed.

### PRIORITY CLAIM

This application is a continuation of International Application Serial No. PCT/US2004/040425, filed Dec. 2, 2004, which claims the benefit of U.S. Provisional Application Ser. No. 60/526,176, filed Dec. 2, 2003. Priority to both of these applications is claimed, and both of the applications are incorporated herein by reference.

### FIELD OF THE INVENTION

The present invention is generally in the field of optics, specifically for measuring aberrations in the eye.

### BACKGROUND OF THE INVENTION

Several refractive defects can occur in the human eye. Refractive defects prevent rays of light entering the eye from properly focusing into a clear image on the retina of the eye. When an eye is focusing properly, a beam of parallel light rays entering the eye will converge to a single point of light on the retina. When an eye cannot do this, because it has refractive errors, the light does not come to a point at the retina, and instead forms a diffuse blob on the retina. These refractive defects may cause a variety of vision problems. Accordingly, measuring and correcting refractive defects in the human eye is of vital importance.

For many years, essentially only two refractive defects, sphere (or “power”) and cylinder (or “astigmatism”), were measurable and correctable. If the eye has too much sphere, light focuses too early within the eye (i.e., in front of the retina), causing myopia or nearsightedness of the eye. If the eye has too little sphere, light focuses too late (i.e., behind the retina), causing hyperopia or farsightedness of the eye. If the eye has a cylinder defect, or an astigmatism, the optics of the eye are “pinched,” such that rays of light from an external source do not converge at a single point on the retina. Sphere and cylinder defects have long been correctable through the use of refractive lenses, and more recently, through refractive eye surgery or other corrective procedures. For ease of description, only vision correction via refractive lenses will be discussed, but the same principles apply for other refractive corrections.

In the case of myopia, the eye refracts, or bends, light too much, causing the light to come to a focus point too early. At this focus point, the light should hit the retina, but since it still has a ways to go, the light crosses over and begins to diverge. By the time the light finally reaches the retina, it has diverged into a diffuse blob, resulting in an image this is not as sharp as it could be.

To correct for myopia, lenses are provided that spread light apart, or cause it to diverge, before the light enters the eye. When a parallel set of light beams passes through such a lens, the beams are no longer parallel, but instead move away from each other at a small angle. The myopic eye is thus provided with light that requires an extra amount of convergence, or focusing, to bring it to a point on the retina. The refraction that the myopic eye applied to the incoming light without a corrective lens was too much, but once the light is diverged by the correcting lens, the refraction that the eye applies to the light is appropriate. Thus, the light is focused at a single point on the retina, without crossing over and diverging again.

In the case of hyperopia, the reverse of myopia is true. The eye does not refract, or bend, the light enough. Thus, the light entering the eye encounters the retina before it has converged to a single point. To correct for hyperopia, lenses are provided that bring the light together, or cause it to converge. This gives the light entering the eye a “head start” on converging, which, coupled with the amount of converging provided by the eye itself, is sufficient to bring the light into a point of focus at the retina.

Developments in optics technology have advanced the understanding of the human eye's function, leading to the discovery of a variety of complex refractive defects in the eye, beyond the basic sphere and cylinder defects. These complex refractive defects are commonly referred to as “higher order aberrations.” Three of the more prevalent higher order aberrations are coma, foils, and spherical aberration.

If the eye has a coma defect, light comes into focus off-center on the retina, leading to vision distortion. If the eye has a foil defect, the outer edges of the eye's optics are wavy, causing poor vision under darkened viewing conditions. If the eye has a spherical aberration, light focus gets worse as viewing conditions become darker and the pupil opens up. As the pupil opens, the light begins to focus in front of the retina. The more the pupil opens up, the farther in front of the retina the light focuses, leading to nearsightedness in darkened conditions. The reverse conditions could also exist, such that the focus point becomes further as the pupil opens.

A device known as a wavefront sensor has been developed to measure these and other aberrations in the eye. In wavefront sensing, a low-power light is projected into the eye. The light that reflects back out of the eye is captured and passed through an analyzer. The analyzer converts the reflected light into patterns or shifts recognizable by a computer. The computer deciphers these patterns or shifts to determine the refractive properties of the eye.

The basic concept of a wavefront sensor is to pass light through the eye in the reverse direction of how the eye normally functions (i.e., to make light come out of the eye rather than enter it), and to analyze any distortions in the emerging light beam. This concept is based on the idea that if a beam of parallel light enters into a well-focused (on infinity) eye, it will be focused into a small spot on the retina. Thus, if a small spot of light is radiated outwardly from the retina of the same good eye (i.e., out of the eye), then the emerging light beam will also be a parallel beam. Conversely, if the exiting beam is converging (i.e., not parallel), it is an indication that the eye is focusing too much, or is myopic. If the exiting beam is diverging, it is an indication that the eye is not focusing enough, or is hyperopic. The exiting beam can be analyzed at many different points to create a complex map of focus powers at various points across the eye.

The first step in the process of wavefront analysis is to create a small point of light on the retina of the eye. The only known feasible way to get light into the eye is through the cornea and lens, which are the same optics that the eye uses to see. Since the eye being examined likely includes refractive errors, the cornea and lens do not focus light well into a small point on the retina, making the job of creating a small point of light on the retina a very difficult task.

Any diffuseness of the light source formed at the retina will cause problems in the return signal, and perhaps an erroneous measurement. For example, a measurement of astigmatism may be reported as higher than actual because not only is the eye astigmatic, but the original light beam that reflected from the retina had some astigmatism in it as well. This false astigmatism was created at the very beginning of the light beam's journey from the retina, because it picked it up on the way into the astigmatic eye.

Attempts have been made to deal with these, and other known problems, by projecting a small spot of light into the center of the cornea where refraction is almost zero. While this method is effective, it is not very forgiving of optical extremes, and is not well-suited for use in combination with a refractive device, as will be described in detail herein.

To create the refractive correction required for higher order aberrations, in addition to the basic refractive errors, adaptive optics or refractive devices, such as electro-active lenses, or “computer programmable lenses,” have been used. Adaptive optics, however, cannot effectively correct for higher order aberrations without first being able to measure them accurately. Meanwhile, the measurement of the higher order aberrations cannot be accurately made without their simultaneous correction.

Thus, there is a need for a wavefront sensor and a refractive correction simulation to work in conjunction with one another, in a single, integrated device. Although there have been several instances where wavefront sensors and refractors have been used in combination, simply combining the two stand-alone devices, without taking into account the effect that the refractive correction device will have on the operation of the illumination beam of the wavefront sensor, will introduce errors. Thus, there is a need for an integrated device that coordinates the functions of a wavefront sensor and a refractor, to improve their collective performance, and to take into account the higher order refractive errors and the changes in them during accommodation.

### BRIEF SUMMARY OF THE INVENTION

The invention involves the integration of a measurement device, such as a wavefront sensor, for measuring higher order aberrations in an eye, with an adaptive optics device, such as an electro-active lens, for correcting the higher order aberrations. This integrated technology, referred to herein as an interactive refractor, not only enables diagnosis of higher order aberrations, but also allows the creation and demonstration of an optical prescription to correct the higher order aberrations. Additionally, because the entire analysis and correction process may take place during an examination sitting, a patient is able to immediately see what effect the correction will have on his/her eyesight.

In one aspect, a broad beam of light (rather than a narrow beam of light that is typically used in wavefront sensing) is directed through an adaptive lens (or lenses), which does not refract the light beam before it enters a patient's eye. The light beam enters the eye, and the eye attempts to focus it to a spot on the retina. Reflected return light from the eye is projected onto one or more gratings, which in turn form shadow patterns from which a camera can form an image. The shadow patterns contain information about the refractive characteristics of the eye. The camera's image of the shadow patterns is digitized into a computer. The shadow pattern is then analyzed by a computer program. The point of light that is created within the relay lens system may also be measured for size, with a reduction in size being directly translated into an improvement in the visual performance of the eye.

Refractive defects, including higher order aberrations, in the eye are determined from analysis of the shadow patterns. The computer instructs the adaptive lens to correct these defects, and the entire process is repeated over and over again until an optimal point of light is focused at the retina. The patient may also participate by observing and influencing the quality of the real world image they see being formed on the retina to help make it better. A prescription may then be created from this analysis to correct a patient's vision.

Further embodiments, including modifications, variations, and enhancements of the invention, will become apparent. The invention resides as well in sub combinations of the features shown and described.

### BRIEF DESCRIPTION OF THE FIGURES

In the drawings, wherein the same reference number indicates the same element throughout the several views:

FIG. 1 is a schematic diagram of an interactive refractor according to one preferred embodiment.

FIG. 2 is a schematic diagram of an interactive refractor according to an alternative embodiment.

FIG. 3 is a schematic diagram of an interactive refractor using an alternative wavefront sensor, such as a non-Talbot Moire wavefront sensor.

FIG. 4 is a schematic diagram of an interactive refractor according to another alternative embodiment.

### DETAILED DESCRIPTION OF THE INVENTION

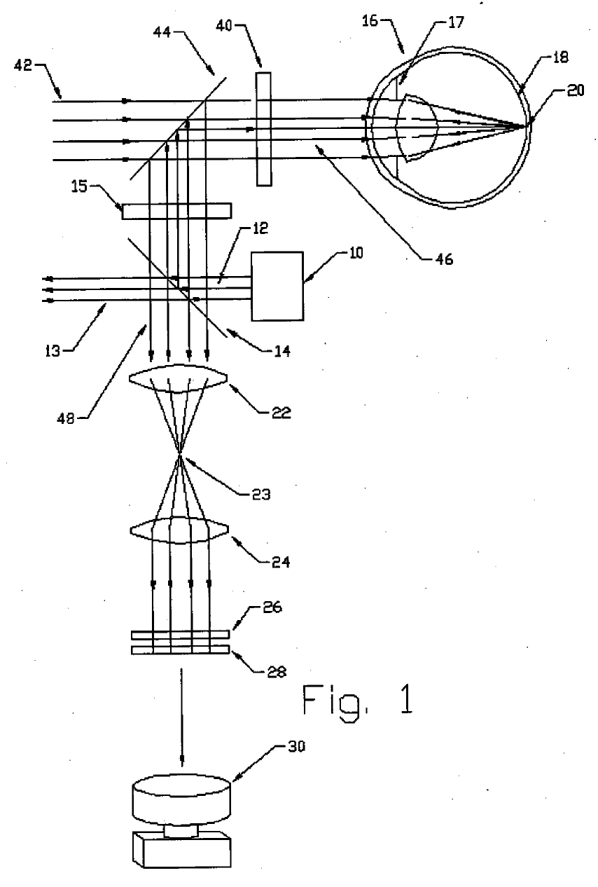

FIG. 1 is a schematic illustration of an interactive refractor system according to a preferred embodiment. The interactive refractor system includes a light source 10, such as a laser generator, for directing a broad, collimated light beam 12 toward a beam splitter 14. The light source 10 preferably generates a light beam 12 having a wavelength of 500 to 1000 nm, more preferably 700 to 800 nm, more preferably 770 to 790 nm. The diameter or cross-sectional area of the beam is preferably at least as large as the area of the surface of the eye to be measured. For example, if the refractive properties of the central 3 mm diameter of the eye are to be measured, then the beam should be at least 3 mm in diameter, and be incident upon the 3 mm diameter of the eye.

At approximately 780 nm, the light beam 12 is virtually invisible (other than a small, central spot) to the human eye, which provides for optimal analysis, since the eye does not react to the entering light. If visible light of shorter wavelengths were used, the eye's pupil and accommodation characteristics would come into play, interfering with the measurement. Higher and lower wavelengths may additionally or alternatively be used, however, to test the eye's reaction to different wavelengths associated with visible light. In this manner, a prescription can be crafted to accommodate the eye's reaction to different colors, or different wavelengths of light.

The beam splitter 14 reflects a first portion of the light beam 12 toward a combiner 44, and allows a second portion 13 of the light beam 12 to pass through the beam splitter 14. The beam splitter 14 preferably has a transmission/reflection ratio of approximately 90/10, but could have any other suitable transmission/reflection ratio.

The combiner 44, is preferably a “hot mirror” (i.e., a mirror that reflects IR light, but passes visible light), or a beam splitter, that is preferably 100% reflective to the laser light beam 12, but 100% transmissive to real-world image light 42. The combiner 44 reflects the first portion of the light beam 12 toward an adaptive optics device or adaptive lens 40. The real-world image light 42 passes through the combiner 44 and joins the reflected light beam 12, forming a combination light beam 46 directed toward the adaptive optics device 40.

The adaptive optics device 40 is preferably spaced from a patient's eye 16 by a distance equal to a distance that a corrective lens, such as a spectacle glass or a contact lens, would be spaced from the eye 16 when a patient wears the corrective lens. Accordingly, a calibration is not required to compensate for the spacing of the adaptive optics device 40 relative to the patient's eye. One or both sides of the adaptive optics device 40 are preferably coated with an anti-reflective material, such as magnesium fluoride, or another suitable material, to prevent unwanted reflections, or “glint,” that could interfere with the analysis process.

The adaptive optics device 40 is preferably a see-through electro-active lens capable of creating higher order refractions as light passes through or reflects from it. The electroactive lens bends the light that passes through or reflects from it, with the amount of bending being varied in a controlled manner, and with electricity being part of the control mechanism or system.

Other systems capable of creating higher order refractions may alternatively be used. For example, deformable mirrors, micro-mirror arrays, physically deformable lenses, alterable-index-of-refraction lenses, or any other suitable devices may be used, but a see-through electro-active lens is preferred. If other lens systems are used that are not “see-through,” and/or cannot be positioned in the same location that the spectacle lenses being prescribed will be placed, then additional optics may be required to compensate for the position shift. Such additional optics designs are known to those skilled in the art of ophthalmic and optic design.

An electro-active lens varies its focus power according to how much electricity is applied to it. Applying more electricity to the electro-active lens causes a higher index of refraction (i.e., more light-bending power). A number of individually controllable smaller lenses is preferably assembled within one larger lens, allowing the refractive power of the electro-active lens to be adjusted at each individual smaller lens's location. Accordingly, complex and detailed refractive prescriptions, covering several individual regions of a patient's eye, can be created.

The combination light beam 46 exiting the combiner 44 passes through the electro-active lens 40, which does no refracting at this time, and enters a patient's eye 16. If the eye 16 exhibits optimal focus, a tiny point of light 20 will form at the retina 18 of the eye 16. If the eye 16 does not exhibit optimal focus, however, the point of light 20 will not properly focus on the retina 18.

A return beam 48 of light, or a wavefront, reflects out of the eye 16, passes back through the electro-active lens 40, and reflects off of the combiner 44 toward the beam splitter 14. Most of the return beam 48 passes through the beam splitter 14 toward one or more relay lenses, such as a first telescopic lens 22, and a second telescopic lens 24. The first and second telescopic lenses 22, 24 are preferably set up to focus the return beam 48 onto a first grating 26. The return beam 48 passes through the first grating 26, preferably to a second grating 28. A greater or lesser number of gratings may alternatively be used.

The first and second gratings 26, 28 preferably include solid lines etched onto a glass substrate at 90 degree angles. The period between each pair of lines is preferably approximately 1 to 250 microns, more preferably 20 to 30 microns, more preferably 25.4 microns. In a preferred embodiment, the lines are solid with distinct edges, but sinusoidal lines or other patterns may also be used. Between each pair of solid lines is preferably a clear line of substantially equivalent width to the solid or sinusoidal lines.

After the return light beam 48 passes through the second grating 28, an image of the return beam 48, or wavefront, is formed as shadow patterns in a space slightly below the second grating 28. The shadow patterns, formed by the one or more gratings 26, 28, contain information about the refractive characteristics of the eye. A camera 30, such as a CCD camera, records the shadow patterns onto a CCD chip or similar device. The image is then digitized into a computer for analysis by a computer program, as is known in the art. As a result, the refractive power of the eye 16 can simultaneously be measured at several points across the pupil 17.

The shadow patterns may be directed to the CCD chip via optics, or they may be formed directly on the CCD chip by placing the CCD chip at a Talbot plane, as is known in the art. Additional analysis may be conducted at this point to gauge the quality of the point of light 20 being formed on the retina, and to measure the contrast of the fringes being formed by the reticle 26, or reticles 26 and 28. The higher the contrast of the fringes, the smaller, or better, the point of light 20 will be. Contrast measurement is known to those skilled in the art of computer image analysis.

Once the computer analysis of the return signal is completed, a coarse approximation of the refractive error of the eye is made. The adaptive lens 40 (or lenses), is then adjusted (or replaced with a lens of different poser), in the direction to refract light in the manner best known to improve the eye's ability to focus. This could be either to diverge the light (for near-sighted myopes), converge the light (for far-sighted hyperopes), or diverge and/or converge different amounts along different axes (for astigmatism, or cylinder).

When the adaptive lens 40 is adjusted, the real-world image light 42 enters the eye at the same time the illumination light beam from the wavefront sensor is projected into the eye, and they are refracted simultaneously. From the light gathered from the real-world image, the eye forms a visual image that the patient can see. The image will now appear better to the patient. From the illumination beam of the wavefront sensor, the eye 16 forms a smaller spot of light on the retina 18 than it did previously, and the reflected signal causes an improved signal for the wavefront sensor to measure. The adaptive lens 40 is performing both the tasks of refracting the wavefront sensor broad-illumination-beam, and refracting the visual image light. If there is a difference between the refractive power of the adaptive lens 40 in the visible light being seen by the patient, and the infrared light being used by the wavefront sensor, then a compensator lens 15 may be used to refract the infrared light, more or less, such that the differences can be neutralized.

If the adjustment made to the adaptive lens 40 was correct, both the image observed by the patient, and the quality of the wavefront signal will improve. If no improvement is detected, or the vision and return signal become worse, then the adaptive lens 40 must be adjusted in the reverse direction. From a series of measurements taken over a range of known refractive conditions that cross the range of “too-much” and “too-little” correction, a fairly accurate best-correction of sphere and cylinder becomes known. At this fairly accurate best-correction point, a more detailed analysis is performed on the returning wavefront beyond coarse approximations and sphere and cylinder measurements.

In the case of a wavefront sensor being of the preferred Talbot and/or Moire system, the improvement of the quality of the wavefront signal may also be observed by an increase in contrast in the fringe patterns observed. Furthermore, the size of the minimum spot at the crossover point 23 will become smaller, and can be measured by various sensors known to those skilled in the art of optics.

The higher order aberrations are quantified at this point and the adaptive lens 40 is again adjusted, but at this point in the process more subtle changes are made (i.e., to correct the higher order aberrations). The measurement-adjust-measurement process is repeated in a closed loop as many times as required until no further improvement in the returning wavefront is detected. Once the desired point of light is created, a final analysis is made by the computer to determine whether there are any subtle refractive errors remaining in the eye 16, and if so, the electro-active lens 40 is further fine-tuned to eliminate those remaining errors. This entire process may take only seconds.

Once an optimal point of light is formed, an optical prescription may be created based on the refractive correction information provided by the adaptive lens 40. This prescription may then be used to craft a detailed refractive lens having several unique refractive regions, or as a guide or “map” to follow when performing refractive surgery on the patient's eye. Additionally, because the entire refractive process may take place during a single examination sitting, a patient is able to immediately see what effect the correction will have on his/her eyesight.

By combining the wavefront sensing equipment and the electro active lens 40 into a single integrated device, the resulting interactive refractor is less complex, more accurate, less costly, more efficient, faster, and more robust than simply using two such devices together without the integration disclosed herein.

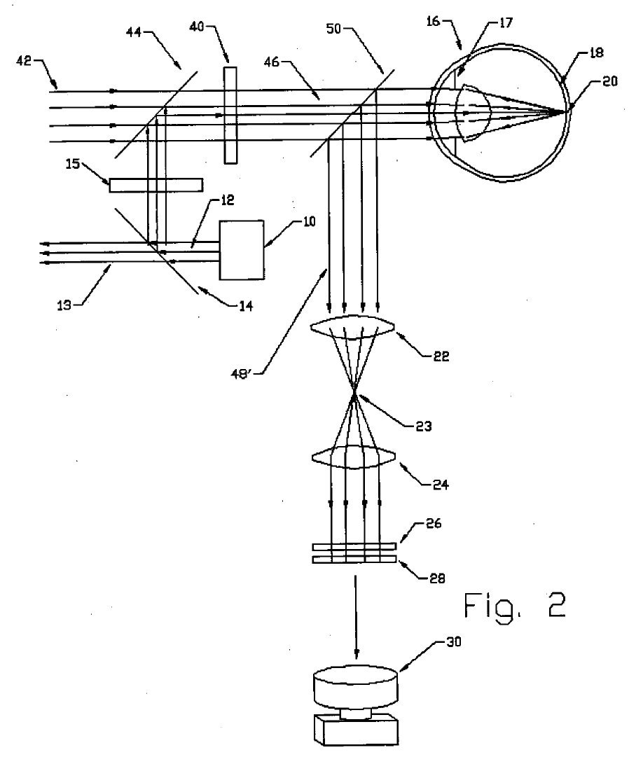

As illustrated in FIG. 2, in an alternative embodiment, a second beam splitter 50 is positioned between the adaptive lens 40 and the eye 16 for intercepting the return beam of light before it passes back through the adaptive lens 40. In this configuration, the detection elements of the optics system (elements 22 through 30) receive only the light 48′ emerging from the eye 16, and not any unwanted portion of light, or “glint,” that may reflect from the adaptive lens 40 when the light beam hits it (again, in the embodiment illustrated in FIG. 1, the adaptive lens 40 is preferably coated with an anti-reflective material to prevent any unwanted light from reaching the detection elements of the optics system).

This positioning of the second beam splitter 50 between the adaptive lens 40 and the eye 16 may cause the adaptive lens 40 to be spaced farther from the eye 16 than an actual corrective lens would be spaced from the patient' eye 16. In such a case, a calculation must be performed to calculate where an actual corrective lens would be positioned relative to the patient's eye 16. These calculations are known to those skilled in the art of optics and ophthalmics.

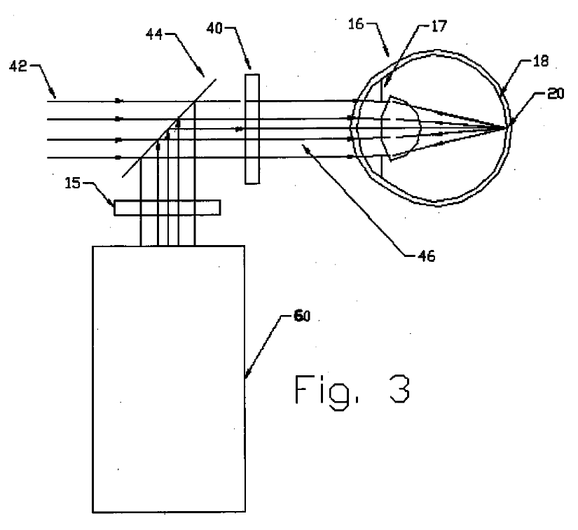

FIG. 3 illustrates an alternative embodiment wherein an alternative wavefront sensor 60, such as a non-Talbot Moire wavefront sensor, is used. Some non-limiting examples of alternative wavefront sensors are Hartmann Shack, OPD, Ray Tracing, and Tscherning, all of which are known by those skilled in the art of wavefront sensing.

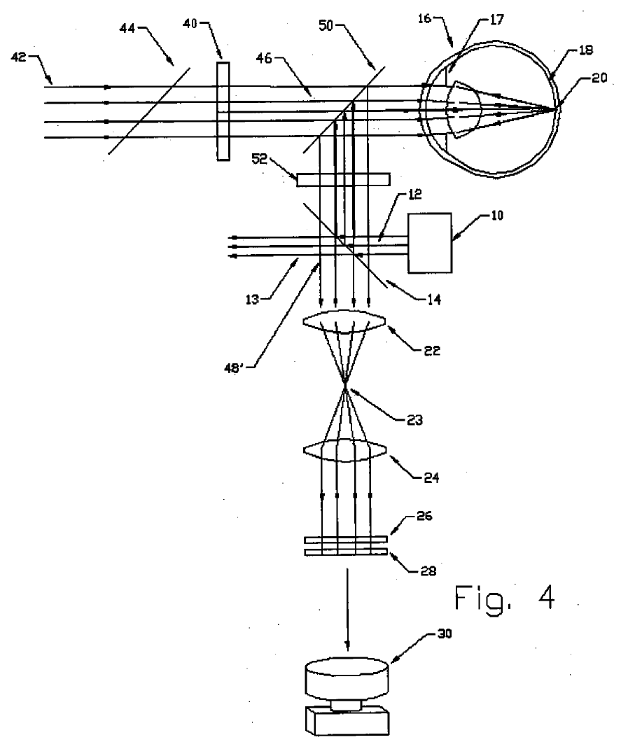

FIG. 4 illustrates an alternative embodiment using a second electroactive lens 52, which controls only the shaping of the illumination beam and return signal for the wavefront sensor. This configuration offers another method of compensating for the differences in refraction between the different wavelengths used in real world images (visible light), and the wavefront sensor's illumination beam (typically Infrared light). The differences in refraction (chromatic differences) are taken into account by having the two electroactive lenses work in conjunction to achieve the same result of focus on the eye. For example, if visible light refracts 10% more than infrared light in the electroactive lenses, then the second electroactive lens 52 would be set to refract 10% less so that the actual amount of refraction would be equal between the two lenses.

The invention disclosed herein, which integrates the two processes of measuring and correcting the eye's refraction into a single device, creates a reliable, undistorted point source of light on the retina of the eye. As a result, higher order aberrations in the eye may be corrected to a great degree of precision. Additionally, the invention overcomes the problems faced by others trying to form a reliable point source of light on the retina through the use of a small diameter light beam and a stand-alone wavefront sensor, which only measures aberrations in the eye, and does not correct refractive errors in the eye.

The invention takes advantage of the refractive properties of the prescription lens being used to improve the vision of the patient, and to also refract the illumination beam of the wavefront sensor to help it form a point source of light on the retina. Additionally, by measuring the quality of the point source of light on the retina (quality of the point source is defined as the “smallest” point), as the prescription lens is being altered, the direct impact of the change in the prescription lens on the focus of the eye may be made without intermediate mathematical interpretation. Such intermediate mathematical interpretation typically adds calculation time, cost, and potential for error to the measurement process.

In other words, the disclosed system emphasizes measuring the quality of the point of light formed on the retina. It utilizes the refractive correction lens, or the adaptive lens to not only refract the light that enters the eye for imaging, but also to refract the light that creates the point source of light. The basic premise is, what is effective at forming a point of light is also effective at correcting the refraction, and vice versa. Rather than only analyze the wavefront emerging from the eye, the disclosed system also measures how small of a spot is being formed on the retina by the prescription lens. The smaller the spot, the better the prescription. Once the smallest spot possible is created, the wavefront sensor measurement aspect is used to perform the final, most sensitive measurement with the confidence that the measurement will not be falsely influenced by a defective originating light point.

Simply measuring the eye's higher order aberrations is not enough to do the job properly. As the eye focuses, or “accommodates,” the higher order aberrations change. The measurement of the higher order aberrations must be made while the eye is in best focus. In other words, if the eye is at rest, gazing outward but not focused on anything, the higher order aberrations will be different than when the eye is focused on an object. Furthermore, the higher order aberrations can be different when the eye is focused on a near object versus a far object. Integrating the adaptive optics with the wavefront sensor allows the measurements and corrections to be made while the eye is in best focus.

When a standalone wavefront sensor is used (without combination with a refraction device), a small diameter beam of light is projected into the eye, or alternatively, great efforts are made to “clean up” the return reflection in the case of a larger diameter beam of light. This is done because any refractive error in the eye deteriorates the quality of the illumination beam going into the eye, and hence, the return signal reflected back as well, making measurement difficult. In the wavefront sensor in this invention, a large beam is projected in an attempt to actually produce a poor return signal, which indicates a refractive problem in the eye. A poor return signal is characterized by low contrast shadow patterns. In the case of an eye having refractive error, the large beam of light projected into the eye would not come into a small point of focus, and instead would form a more diffuse spot.

By constructing the electro-active lens (or lenses) in an array of many individually-controllable cells, each cell may be uniquely programmed as to how much refractive power it contributes to the entire lens, but only within its small area. Such an arrangement overcomes the previous constraints of having only one refractive power along each meridian of a lens, allowing the production of highly complex refractive corrections capable of correcting previously uncorrectable higher order aberrations.

Thus, while several embodiments have been shown and described, various changes and substitutions may of course be made, without departing from the spirit and scope of the invention. The invention, therefore, should not be limited, except by the following claims and their equivalents.

What is claimed is:

1. An apparatus for measuring refraction of an eye, comprising:
a wavefront sensor for producing an illumination beam at least as large as an area of the eye being measured;
an electroactive lens operatively coupled with the wavefront sensor, such that both the illumination beam produced by the wavefront sensor, and real world light entering the eye, are refracted by the electroactive lens;
wherein the wavefront sensor makes adjustments to the electroactive lens, which are observable by the eye, to measure higher order aberrations in the eye.

2. An apparatus for measuring refraction of an eye, comprising:
a wavefront sensor for producing an illumination beam at least as large as an area of the eye being measured;
an electroactive optic operatively coupled with the wavefront sensor, such that both the illumination beam produced by the wavefront sensor, and real world light entering the eye, are reflected by the electroactive optic;
wherein the wavefront sensor makes adjustments to the electroactive optic, which are observable by the eye, to measure higher order aberrations in the eye.

3. An apparatus for measuring refraction of an eye, comprising:
a wavefront sensor for producing an illumination beam at least as large as an area of the eye being measured;
a first electroactive lens operatively coupled with the wavefront sensor for modifying the illumination beam produced by the wavefront sensor;
a second electroactive lens operatively coupled with the wavefront sensor for modifying a wavefront of light entering the eye to form real world images;
an aperture-sharing element operatively coupled with the first and second electroactive lenses for combining the wavefront of light entering the eye to form real world images into a common path with the modified illumination beam before they enter the eye;
wherein the wavefront sensor makes and coordinates corrections to the first and second electroactive lenses, with the corrections observable by the eye during the refractive procedure, such that a change in higher order aberrations of the eye can be measured and adjusted for.

4. An apparatus for measuring refraction of an eye, comprising:
a wavefront sensor for producing an illumination beam at least as large as an area of the eye being measured;
an electroactive lens operatively coupled with the wavefront sensor for modifying the illumination beam produced by the wavefront sensor, and for modifying a wavefront of light entering the eye to form real world images;
an aperture-sharing element operatively coupled with the electroactive lens for combining the wavefront of light entering the eye to form real world images into a common path with the modified illumination beam before they enter the eye;
wherein the wavefront sensor makes corrections to the electroactive lens, with the corrections observable by the eye during the refractive procedure, such that a change in higher order aberrations of the eye can be measured and adjusted for.

5. A method for measuring the refraction of an eye, comprising the steps of:
producing, via a wavefront sensor, an illumination beam at least as large as an area of the eye being measured;
refracting the illumination beam and real world light entering the eye with an electroactive lens;
measuring a correction to the electroactive lens via the wavefront sensor; and
adjusting, based on the measured correction, the electroactive lens to account for higher order aberrations in the eye.
